(19) United States
(12) Patent Application Publication
(10) Pub. No.: US 2023/0178241 A1
(43) Pub. Date: Jun. 8, 2023

(54) METHOD OF PREDICTING FETAL OR NEONATAL DISEASE BASED ON STAGE OF MATERNAL PERIODONTITIS

(71) Applicant: Korea University Research and Business Foundation, Seoul (KR)

(72) Inventors: Ki Hoon AHN, Seoul (KR); Ju Sun HEO, Seoul (KR); Jung Soo PARK, Hanam-si (KR)

(21) Appl. No.: 18/073,147

(22) Filed: Dec. 1, 2022

(30) Foreign Application Priority Data
Dec. 3, 2021 (KR) 10-2021-0171440

(51) Int. Cl. G16H 50/20; G16H 70/60

(52) U.S. Cl. CPC G16H 50/20; G16H 70/60

(57) ABSTRACT

A method of predicting fetal or neonatal disease based on the stage of maternal periodontitis is presented to predict fetal or neonatal disease, specifically, any one or more of small for gestational age, neonatal general health, neonatal respiratory disease, retinopathy of prematurity, and patent ductus arteriosus requiring treatment, before birth by determining the stage of maternal periodontitis in a noninvasive and clear manner.

### CROSS-REFERENCE TO RELATED APPLICATION

This application claims the benefit of Korean Patent Application No. 10-2021-0171440 filed on Dec. 3, 2021, in the Korean Intellectual Property Office, the entire disclosure of which is incorporated herein by reference for all purposes.

### BACKGROUND

### 1. Technical Field

The present invention relates to a method of predicting fetal or neonatal disease based on the stage of maternal periodontitis.

### 2. Related Art

Pregnancy is a period in which physical changes occur rapidly along with hormonal changes in vivo. In addition, it is known that there are many changes in the periodontal tissue in the oral cavity, and when women of childbearing age become pregnant, gestational gingivitis occurs in 35 to 100% of the women, about 10% of which may lead to pyogenic granuloma. The reason why these changes in the periodontal tissue appear is known to be because the concentrations of estrogen and progesterone increase due to pregnancy, and the synthesis of prostaglandins, which are fatty acids that play an important role in biological processes such as vasodilation, platelet aggregation inhibition, and inflammation, is mainly made in the tissues in the gingival sulcus, causing gingival hemorrhage and changes in the subgingival flora. In addition, pregnancy is a period in which the sensitivity to inflammation may increase, increasing the risk for periodontal tissue infection. Although the occurrence time or severity of periodontal tissue changes in pregnancy has been reported differently for each study, it is generally known that the expression of inflammation in the second and third trimesters of pregnancy is higher than that in early pregnancy.

Since the mother, fetus, and newborn are inseparable from each other, good health during pregnancy affects the growth and development of the fetus as well as the health of the mother, and lowers the risk or probability of fetal or neonatal disease.

However, the correlation between maternal periodontitis and the fetus or newborn, in particular, the correlation between maternal periodontitis and diseases such as small for gestational age, which can be fatal to the fetus or newborn, neonatal general health, neonatal respiratory disease, retinopathy of prematurity, and patent ductus arteriosus requiring treatment, remains unknown.

### SUMMARY

An object of the present invention is to provide a method of predicting fetal or neonatal disease based on the stage of maternal periodontitis, the method comprising steps of: a) determining the stage of maternal periodontitis based on criteria shown in Table 1; and b) predicting fetal or neonatal disease based on the stage of maternal periodontitis determined in step a).

One aspect of the present invention provides a method of predicting fetal or neonatal disease based on the stage of maternal periodontitis, the method comprising steps of: a) determining the stage of maternal periodontitis based on criteria shown in Table 1 below; and b) predicting fetal or neonatal disease based on the stage of maternal periodontitis determined in step a):

TABLE 1

| Periodontitis Stage | | Stage I | Stage II | Stage III | Stage IV |
|---|---|---|---|---|---|
| Severity | Interdental | 1 to 2 mm | 3 to 4 mm | ≥5 mm | ≥5 mm |
| | clinical | | | | |
| | attachment | | | | |
| | loss at site of | | | | |
| | greatest loss | | | | |
| | Radiographic | Coronal | Coronal | ⅓ to ⅔ or | ⅓ to ⅔ or |
| | vertical bone | third (<15%) | third (15% | more of | more of |
| | loss | | to 33%) | tooth root | tooth root |
| | Whether | No tooth loss due to | | Loss of 4 or | Loss of 5 or |
| | teeth are lost | periodontitis | | less teeth | more teeth |
| | | | | due to | due to |
| | | | | periodontitis | periodontitis |
| Complexity | Local area | Maximum | Maximum | In addition | In addition to |
| | basis | probing | probing | to Stage II | complexity |
| | | depth ≤4 mm | depth ≤5 mm | complexity: | of stage II: |
| | | Mostly | Mostly | Maximum | Need for |
| | | horizontal | horizontal | probing | complex |
| | | bone loss | bone loss | depth ≥6 mm | rehabilitation |
| | | | | Vertical bone | due to: |
| | | | | loss ≥3 mm | masticatory |
| | | | | Furcation | dysfunction |
| | | | | involvement | Secondary |
| | | | | Class II or III | occlusal |
| | | | | Moderate | trauma (tooth |
| | | | | alveolar | mobility of 2 |
| | | | | ridge defect | degrees) |
| | | | | | Severe |
| | | | | | alveolar |
| | | | | | ridge defect |
| | | | | | Bite |
| | | | | | collapse |
| | | | | | Less than |
| | | | | | 20 remaining |
| | | | | | teeth |

Step a) is a step of determining the stage of maternal periodontitis based on the criteria shown in Table 1 above.

In one embodiment of the present invention, the determining in step a) may be performed based on a radiograph of the mother's teeth. Specifically, the radiograph may be a panoramic radiograph, and may be a dental radiograph before childbirth considering the purpose and effect of the present invention.

The stage of maternal periodontitis is determined based on the criteria shown in Table 1 above, and the stages of maternal periodontitis (Stages I to IV) are not unclearly determined by the conventional arbitrary judgment of doctors, but are determined based on the clear criteria. In the present invention, Stage I also includes a normal group and is classified as mild/moderate periodontitis (MP) together with Stage II, and Stages III and IV are classified as severe periodontitis (SP).

Step b) is a step of predicting fetal or neonatal disease based on the stage of maternal periodontitis determined in step a).

Specifically, if the stage of maternal periodontitis is Stage III or IV, that is, severe periodontitis, it may be predicted and determined that the likelihood of development of developing fetal or neonatal disease is high.

In another embodiment of the present invention, the fetal or neonatal disease in step b) may be any one or more of small for gestational age, neonatal general health, neonatal respiratory disease, retinopathy of prematurity, and patent ductus arteriosus requiring treatment.

In the present invention, “determining” the likelihood of developing maternal, fetal or neonatal disease has substantially the same meaning as “predicting” the likelihood.

In the present invention, the method may further comprise a step of determining that, when the stage of maternal periodontitis determined in step a) corresponds to Stage III or Stage IV, the likelihood of very preterm birth with a gestational age (GA) of 32 weeks or less or extremely preterm birth with a gestational age (GA) of 28 weeks or less is higher than that when the stage of maternal periodontitis corresponds to Stage I or Stage II.

In the present invention, the method may further comprise a step of determining that, when the stage of maternal periodontitis determined in step a) corresponds to Stage III or Stage IV, the likelihood of developing fetal or neonatal disease is higher than that when the stage of maternal periodontitis corresponds to Stage I or Stage II.

In the present invention, the method may further comprise a step of determining that, when the stage of maternal periodontitis determined in step a) corresponds to Stage I or Stage II, the likelihood of small for gestational age is 0.1 to 5%.

In the present invention, the method may further comprise a step of determining that, when the stage of maternal periodontitis determined in step a) corresponds to Stage III, the likelihood of developing small for gestational age is 20 to 25%.

In the present invention, the method may further comprise a step of determining that, when the stage of maternal periodontitis determined in step a) corresponds to Stage IV, the likelihood of small for gestational age is 30 to 35%. Characteristics related to the likelihood of small for gestational age are based on the contents of FIG. 3 and Examples.

In the present invention, the method may further comprise a step of predicting that, when the stage of maternal periodontitis determined in step a) corresponds to Stage III or Stage IV, the duration of respiratory support of the newborn will be longer and/or the Apgar score of the newborn will be higher than that when the stage of maternal periodontitis corresponds to Stage I or Stage II.

In the present invention, the method may further comprise a step of predicting that, when the stage of maternal periodontitis determined in step a) corresponds to Stage III or Stage IV, the likelihood of Caesarean section will be higher than that when the stage of maternal periodontitis corresponds to Stage I or Stage II.

In the present invention, the method may further comprise a step of predicting that, when the stage of maternal periodontitis determined in step a) corresponds to Stage III or Stage IV, the likelihood of maternal uterine ischemia, chronic hypertension, preedampsia, and/or uterine leiomyoma will be higher than that when the stage of maternal periodontitis corresponds to Stage I or Stage II.

In the present invention, the method may further comprise a step of predicting that, when the stage of maternal periodontitis determined in step a) corresponds to Stage III or Stage IV, the birth weight of the newborn or the Z-score of the birth weight will be lower than that when the stage of maternal periodontitis corresponds to Stage I or Stage II.

According to the method of predicting fetal or neonatal disease according to the present invention, it is possible to predict fetal or neonatal disease, specifically, any one or more of small for gestational age, neonatal general health, neonatal respiratory disease, retinopathy of prematurity, and patent ductus arteriosus requiring treatment, before birth by determining the stage of maternal periodontitis in a noninvasive and clear manner.

### BRIEF DESCRIPTION OF THE DRAWINGS

FIG. 1 shows a process of selecting a study population in the present invention.

FIG. 2 shows panoramic radiographs of the mothers used in the study in the present invention.

FIG. 3 is a graph showing the incidence of small for gestational age (SGA) according to the stage of maternal periodontitis.

### DETAILED DESCRIPTION

Hereinafter, one or more embodiments will be described in more detail with reference to examples. However, these examples are for explaining one or more embodiments, and the scope of the present invention is not limited to these examples.

### Example 1: Study Method

Study Design and Population

The study subjects were mothers who underwent panoramic radiography within 5 years before and after the time of delivery in the Korea University Anam Hospital between Jan. 1, 2010, and Dec. 12, 2019. Panoramic radiographs were taken when the subjects had visited the Department of Dentistry, Anam Hospital for wisdom tooth extraction or dental treatment. Cases involving stillbirth or multiplets were excluded. The medical records of mothers included in the study and their newborns were retrospectively reviewed.

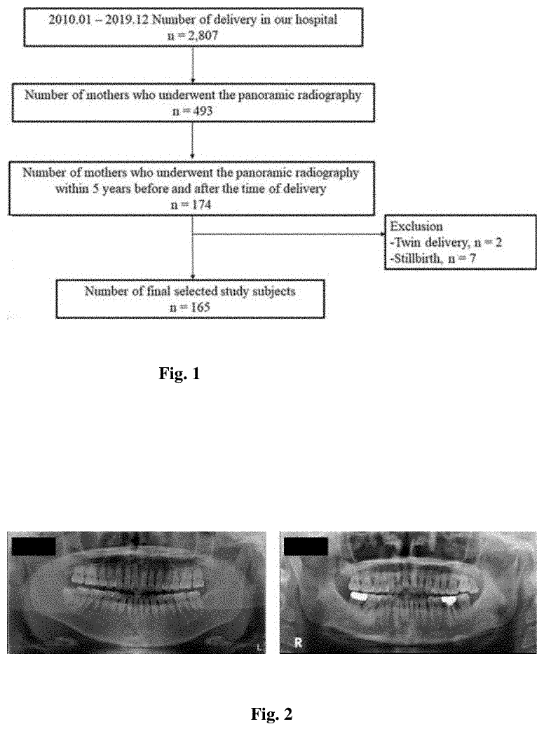

A total of 2,807 mothers gave birth during the study period (FIG. 1). Among them, 493 mothers (17.6%) underwent panoramic radiography, and 174 mothers (6.2%) underwent panoramic radiography within 5 years before and after the time of delivery. Among the 174 mothers, two mothers who gave birth to twins and seven mothers with stillbirth were excluded from this study. A final study population of 165 mothers and their neonates was included in this study.

Clinical Data Collection

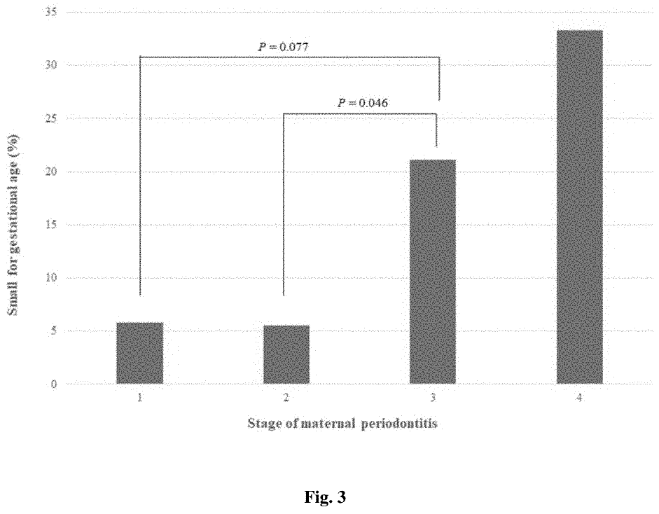

The present inventors used a new classification of periodontal disease (see Table 1) introduced following consensus reports of an International Workshop that took place in November 2017 to assign moderate periodontitis (MP) or severe periodontitis (SP) in this study. MP refers to Stage I or II periodontitis, while SP refers to Stage III or IV periodontitis. Intra-examiner reliability was validated using kappa coefficients from 100 panoramic radiographs before the study (Cohen's kappa value: 0.90) (FIG. 2). However, periodontally healthy cases fell into Stage I because it is impossible to discern healthy cases from Stage I without the help of full-mouth periodontal examination records.

The diagnosis of periodontitis was based on radiographs taken within a 5-year period before and after delivery. Periodontitis is a chronic inflammatory disease that affects the tissues surrounding the teeth. Regardless of the disease stage, it is widely known that disease progression rates do not exceed 0.15 mm/year in terms of mean annual alveolar bone loss. This indicates that the findings observed 5 years prior to or following delivery sufficiently reflect the patient's periodontal status during pregnancy.

Here, small for gestational age (SGA) was defined as birth weight below the 10th percentile according to the Fenton growth chart. In addition, a study was also conducted on diseases such as fetal retinopathy of prematurity and patent ductus arteriosus requiring treatment.

### Example 2: Study Results

Maternal Characteristics and Morbidities in the Severe Periodontitis (SP) and Mild/Moderate Periodontitis (MP) Groups.

Of the 165 mothers enrolled, 22 (13.3%) had SP and 143 (86.7%) had MP. Table 2 below shows the baseline characteristics and morbidities of the mothers according to the severity of periodontitis.

TABLE 2

| | Severe | | Mild/moderate | | |
|---|---|---|---|---|---|
| | periodontitis | | periodontitis | | |
| Factors | n = 22 | | n = 143 | | P-value |
| Age (year) | 34 | (30, 38) | 31 | (29, 34) | 0.068 |
| Age ≥35 years | 8 | (36.4) | 34 | (23.8) | 0.291 |
| Nulliparous | 9 | (40.9) | 81 | (56.6) | 0.177 |
| Height (cm) | 163 | (158, 167) | 161 | (157, 165) | 0.267 |
| Pre-pregnancy weight (kg) | 56 | (52, 66) | 54 | (50, 58) | 0.121 |
| Pre-pregnancy body mass index (kg/m²) | 21.5 | (20.1, 24.2) | 20.8 | (19.3, 22.3) | 0.092 |
| Weight gain during pregnancy (kg) | 12.5 | (8.7, 15.3) | 13.0 | (10.5, 16.1) | 0.378 |
| Body mass index change during pregnancy (kg/m²) | 5.1 | (3.2, 5.6) | 5.0 | (3.9, 6.1) | 0.301 |
| In-vitro fertilization | 1 | (4.5) | 4 | (2.8) | 0.516 |
| Cesarean section | 16 | (72.7) | 73 | (51.0) | 0.068 |
| Prior preterm birth | 2 | (9.1) | 5 | (3.5) | 0.235 |
| Preterm labor | 3 | (13.6) | 20 | (14.0) | 1.000 |
| Prelabor rupture of membrane >18 h | 0 | (0.0) | 16 | (11.2) | 0.134 |
| Chorioamnionitis | 0/11 | (0.0) | 3/56 | (5.4) | 1.000 |
| Funisitis | 0/11 | (0.0) | 0/56 | (0.0) | |
| Pelvic inflammatory disease history | 0 | (0.0) | 1 | (0.7) | 1.000 |
| Uterine leiomyoma | 4 | (18.2) | 6 | (4.2) | 0.029* |
| Type 1 diabetes mellitus | 1 | (5.0) | 0 | (0.0) | 0.125 |
| Gestational diabetes mellitus | 3 | (15.0) | 7 | (5.0) | 0.113 |
| Chronic hypertension | 2 | (9.1) | 1 | (0.7) | 0.047* |
| Preeclampsia | 3 | (13.6) | 3 | (2.1) | 0.032* |
| Thyroid disease | 3 | (13.6) | 27 | (18.9) | 0.768 |
| Chronic medical disease | 6 | (27.3) | 35 | (24.5) | 0.793 |

As can be seen in Table 2 above, maternal, age, pre-pregnancy body mass index (BMI), and incidence of Cesarean section were significantly higher in the SP group than in the MP group.

Uterine leiomyoma, chronic hypertension, and preeclampsia occurred significantly more frequently in the SP group than in the MP group. After adjusting for maternal variables including maternal age≥35 years, pre-pregnancy BMI, and preeclampsia, maternal SP was associated with an increased risk of uterine leiomyoma.

Neonatal Characteristics in the SP and MP Groups

The baseline neonatal characteristics are presented in Table 3 below. There were no significant differences between the groups in terms of gestational age (GA), incidence of preterm birth (GA<37 weeks), sex, or z-scores of birth weight, height, and head circumference. However, the incidences of very preterm birth (GA<32 weeks) and extremely preterm birth (GA<28 weeks) were significantly higher in the SP group than in the MP group. The incidence of small for gestational age (SGA) was significantly higher in the SP group than in the MP group. The incidence of SGA remained low, at around 5%, in those with stage≤II disease but increased rapidly in those with stage≥III disease (FIG. 3). After adjusting for maternal and neonatal variables, including maternal age≥35 years, pre-pregnancy BMI, uterine leiomyoma, preeclampsia, Caesarean section, and GA<32 weeks, maternal SP was associated with an increased risk of SGA (aOR 4.488, 95% CI 1.116-18.058, P=0.035).

TABLE 3

| | Severe | | Mild/moderate | | |
|---|---|---|---|---|---|
| | periodontitis | | periodontitis | | |
| Factors | n = 22 | | n = 143 | | P-value |
| Gestational age (weeks) | 37⁺⁵ | (36⁺⁴, 39⁺⁵) | 38⁺⁵ | (37⁺², 39⁺³) | 0.227 |
| Gestational age <37 weeks | 6 | (27.3) | 20 | (14.0) | 0.122 |
| Gestational age <32 weeks | 3 | (13.6) | 2 | (1.4) | 0.017* |
| Gestational age <28 weeks | 2 | (9.1) | 1 | (0.7) | 0.047* |
| Birth weight (g) | 3175 | (2238, 3528) | 3220 | (2880, 3440) | 0.487 |
| Birth weight z-score | −0.196 | (−1.275, 0.454) | −0.023 | (−0.424, 0.351) | 0.345 |
| Height at birth (cm) | 50.0 | (46.8, 53.0) | 50.0 | (48.0, 51.0) | 0.497 |
| Height at birth, z-score | 0.380 | (−0.819, 1.349) | 0.015 | (−0.457, 0.654) | 0.249 |
| Head circumference at birth (cm) | 33.3 | (31.8, 34.5) | 34.0 | (32.5, 35.0) | 0.221 |
| Head circumference at birth, z-score | −0.507 | (−1.451, 0.306) | −0.264 | (−0.915, 0.402) | 0.215 |
| Small for gestational age | 5 | (22.7) | 8 | (5.6) | 0.017* |
| Male | 15 | (68.2) | 78 | (54.5) | 0.257 |
| Neonatal resuscitation program at delivery room | 7 | (31.8) | 21 | (14.7) | 0.064 |
| Apgar score, 1 min | 9.0 | (6.5, 9.0) | 9.0 | (8.0, 9.0) | 0.279 |
| Apgar score, 5 min | 10.0 | (8.8, 10.0) | 10.0 | (9.0, 10.0) | 0.451 |
| Neonatal intensive care unit admission | 8 | (38.4) | 32 | (22.4) | 0.182 |
| Initial white blood cell count | 10,590 | (7800, 16,300) | 14,000 | (9450, 19,100) | 0.065 |
| Initial C-reactive protein | 0.16 | (0.13, 0.99) | 0.14 | (0.11, 0.48) | 0.458 |

Neonatal Characteristics and Morbidities of Preterm Infants

Among the 26 infants born prematurely, six (23.1%) were born to mothers with SP, and 20 (76.9%) were born to mothers with MP. Table 4 below shows the neonatal baseline characteristics and morbidities of preterm infants according to their mothers' severity of

TABLE 4

| | Severe | | Mild/moderate | | |
|---|---|---|---|---|---|
| | periodontitis | | periodontitis | | |
| Factors | n = 6 | | n = 20 | | P-value |
| Gestational age (weeks) | 32⁺² | (28⁺³, 36⁺²) | 34⁺⁵ | (33⁺⁵, 36⁺³) | 0.268 |
| Gestational age <32 weeks | 3 | (50.0) | 2 | (10.0) | 0.062 |
| Gestational age <28 weeks | 2 | (33.3) | 1 | (5.0) | 0.123 |
| Birth weight (g) | 1475 | (845, 2393) | 2300 | (2040, 2896) | 0.046* |
| Birth weight z-score | −1.166 | (−1.461, 0.400) | 0.251 | (−0.231, 0.539) | 0.083 |
| Height at birth (cm) | 39.8 | (33.8, 49.5) | 46.3 | (44.2, 48.9) | 0.157 |
| Height at birth, z-score | −0.249 | (−1.846, 0.926) | 0.549 | (−0.119, 1.235) | 0.242 |
| Head circumference at birth (cm) | 27.8 | (23.6, 33.3) | 31.3 | (30.1, 33.9) | 0.219 |
| Head circumference at birth, z-score | −0.642 | (−1.714, 0.597) | 0.431 | (−0.639, 0.887) | 0.176 |
| Small for gestational age | 2 | (33.3) | 1 | (5.0) | 0.123 |
| Male | 3 | (50.0) | 10 | (50.0) | 1.000 |
| Neonatal resuscitation program at delivery room | 5 | (83.3) | 7 | (35.0) | 0.065 |
| Apgar score, 1 min | 3.0 | (1.8, 7.3) | 8.0 | (7.0, 9.0) | 0.003* |
| Apgar score, 5 min | 8.0 | (6.0, 9.3) | 9.0 | (9.0, 10.0) | 0.062 |
| Initial white blood cell count | 6690 | (4325, 11177) | 9050 | (7843, 12775) | 0.108 |
| Initial C-reactive protein | 0.16 | (0.09, 0.99) | 0.13 | (0.40, 0.24) | 0.933 |
| Sepsis | 0 | (0.0) | 0 | (0.0) | |
| Respiratory distress syndrome | 3 | (50.0) | 2 | (10.0) | 0.062 |
| Moderate to severe bronchopulmonary dysplasia | 1 | (16.7) | 0 | (0.0) | 0.231 |
| Duration of respiratory support (days) | 18.5 | (0.0, 77.3) | 0.0 | (0.0, 1.5) | 0.046* |
| Duration of total parenteral nutrition (days) | 5.5 | (0.0, 59.3) | 0.0 | (0.0, 0.0) | 0.176 |
| Retinopathy of prematurity | 3 | (50.0) | 0 | (0.0) | 0.008* |
| Treated retinopathy of prematurity | 2 | (33.3) | 0 | (0.0) | 0.046* |
| Treated patent ductus arteriosus | 2 | (33.3) | 0 | (0.0) | 0.046* |
| Necrotizing enterocolitis ≥stage 2 | 2 | (33.3) | 1 | (5.0) | 0.123 |
| Intraventricular hemorrhage | 1 | (16.7) | 3 | (15.0) | 1.000 |
| Isolated intraparenchymal hemorrhage | 1 | (16.7) | 0 | (0.0) | 0.240 |
| Periventricular leukomalacia | 0 | (0.0) | 0 | (0.0) | |

Infants in the SP group were born at an earlier GA than infants in the MP group, but this difference was not significant. The incidence of very preterm birth was borderline significantly higher in the SP group than in the MP group. Infants in the SP group not only had lower birth weights but also lower z-scores of birth weight. There was no significant difference in height or head circumference. The incidence of SGA was higher in the SP group than in the MP group. The duration of respiratory support was significantly longer and the incidences of retinopathy of prematurity and patent ductus arteriosus requiring treatment were significantly higher in the SP group than in the MP group. In addition, it was confirmed that the Apgar score (1 min or 5 min) of small for gestational age immediately after birth, which means the overall health of the newborn, was also significantly higher in the SP group than in the MP group.

### Example 3: Discussion

Taking the above study results together, maternal severe periodontitis increased the risk of small for gestational age (SGA), which was maintained even after adjusting other variables. These characteristics were also found in preterm infants.

In addition, the incidence of retinopathy of prematurity (ROP) and the treatment history of patent ductus arteriosus requiring treatment were higher in the SP group than in the MP group.

Therefore, it is predicted that inflammation due to maternal periodontitis is a major cause for the onset of retinopathy of prematurity and patent ductus arteriosus requiring treatment.

So far, the present invention has been described with reference to the embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

1. A method of predicting fetal or neonatal disease based on the stage of maternal periodontitis, the method comprising steps of:
a) determining the stage of maternal periodontitis based on criteria shown in Table 1 below; and
b) predicting fetal or neonatal disease based on the stage of maternal periodontitis determined in step a):
TABLE 1

| Periodontitis Stage | | Stage I | Stage II | Stage III | Stage IV |
|---|---|---|---|---|---|
| Severity | Interdental | 1 to 2 mm | 3 to 4 mm | ≥5 mm | ≥5 mm |
| | clinical | | | | |
| | attachment | | | | |
| | loss at site of | | | | |
| | greatest loss | | | | |
| | Radiographic | Coronal | Coronal | ⅓ to ⅔ or | ⅓ to ⅔ or |
| | vertical bone | third (<15%) | third (15% | more of | more of |
| | loss | | to 33%) | tooth root | tooth root |
| | Whether | No tooth loss due to | | Loss of 4 or | Loss of 5 or |
| | teeth are lost | periodontitis | | less teeth | more teeth |
| | | | | due to | due to |
| | | | | periodontitis | periodontitis |
| Complexity | Local area | Maximum | Maximum | In addition | In addition to |
| | basis | probing | probing | to Stage II | complexity |
| | | depth ≤4 mm | depth ≤5 mm | complexity: | of stage II: |
| | | Mostly | Mostly | Maximum | Need for |
| | | horizontal | horizontal | probing | complex |
| | | bone loss | bone loss | depth ≥6 mm | rehabilitation |
| | | | | Vertical bone | due to: |
| | | | | loss ≥3 mm | masticatory |
| | | | | Furcation | dysfunction |
| | | | | involvement | Secondary |
| | | | | Class II or III | occlusal |
| | | | | Moderate | trauma (tooth |
| | | | | alveolar | mobility of 2 |
| | | | | ridge defect | degrees) |
| | | | | | Severe |
| | | | | | alveolar |
| | | | | | ridge defect |
| | | | | | Bite collapse |
| | | | | | Less than |
| | | | | | 20 remaining |
| | | | | | teeth |

2. The method according to claim 1, wherein the determining in step a) is performed based on a radiograph of maternal teeth.

3. The method according to claim 1, wherein the fetal or neonatal disease in step b) is any one or more of small for gestational age, neonatal general health, neonatal respiratory disease, retinopathy of prematurity, and patent ductus arteriosus requiring treatment.

4. The method according to claim 1, comprising a step of determining that, when the stage of maternal periodontitis determined in step a) corresponds to Stage III or Stage IV, the likelihood of very preterm birth with a gestational age (GA) of 32 weeks or less or extremely preterm birth with a gestational age (GA) of 28 weeks or less is higher than that when the stage of maternal periodontitis corresponds to Stage I or Stage II.

5. The method according to claim 1, comprising a step of determining that, when the stage of maternal periodontitis determined in step a) corresponds to Stage III or Stage IV, the likelihood of developing fetal or neonatal disease is higher than that when the stage of maternal periodontitis corresponds to Stage I or Stage II.

6. The method according to claim 1, comprising a step of determining that, when the stage of maternal periodontitis determined in step a) corresponds to Stage I or Stage II, the likelihood of small for gestational age is 0.1 to 5%.

7. The method according to claim 1, comprising a step of determining that, when the stage of maternal periodontitis determined in step a) corresponds to Stage III, the likelihood of small for gestational age is 20 to 25%.

8. The method according to claim 1, comprising a step of determining that, when the stage of maternal periodontitis determined in step a) corresponds to Stage IV, the likelihood of developing small for gestational age is 30 to 35%.

9. The method according to claim 1, comprising a step of predicting that, when the stage of maternal periodontitis determined in step a) corresponds to Stage III or Stage IV, the duration of respiratory support of the newborn will be longer than that when the stage of maternal periodontitis corresponds to Stage I or Stage II.
